(19) United States
(12) Patent Application Publication
(10) Pub. No.: US 2003/0158265 A1
(43) Pub. Date: Aug. 21, 2003

(54) Orally administrable pharmaceutical formulation comprising pseudoephedrine hydrochloride and process for preparing the same

(76) Inventors: Ramachandran Radhakrishnan, Bangalore (IN); Nehru Babu Gaddipati, Somerset, NJ (US)

Correspondence Address: KNOBBE MARTENS OLSON & BEAR LLP, 2040 MAIN STREET, FOURTEENTH FLOOR, IRVINE, CA, 92614, US

(21) Appl. No.: 10/096,709

(22) Filed: Mar. 13, 2002

(30) Foreign Application Priority Data
Feb. 20, 2002 (IN) 127/DEL/2002

(51) Int. Cl. A61K 31/137; A61K 9/48; A61K 31/075

(52) U.S. Cl. 514/649; 514/718; 424/452

(57) ABSTRACT

Disclosed are pharmaceutical formulations for oral administration through a soft gelatin capsule drug delivery device, wherein the pharmaceutical formulation, in a preferred embodiment, contains Pseudoephedrine HCl and an expectorant as the active ingredients. The active pharmaceutical ingredient is embedded into an oily matrix. The formulation also includes an expectorant; a surfactant; a suspending agent; and a suspension medium, wherein, in a preferred embodiment, the expectorant is guaifenesin, the surfactant is lecithin, the suspending agent is yellow beeswax, and the suspension medium is soybean oil. In a preferred embodiment, the formulation consists essentially of about 30.5 mg by weight of Pseudoephedrine HCl, about 200 mg by weight of guaifenesin, about 0.1-5.0 mg by weight of yellow beeswax, about 10-15 mg by weight of lecithin; and about 200-300 mg by weight of soybean oil. Also disclosed is a process for preparing the formulation.

### BACKGROUND OF THE INVENTION

1. Field of the Invention

This invention in general relates to orally administrable pharmaceutical formulations and in particular to a pharmaceutical formulation prepared into a soft gelatin capsule containing Pseudoephedrine hydrochloride as one of its active ingredients.

2. Description of the Related Art

Pseudoephedrine hydrochloride is a drug that has serious potential for abuse. This is so because Pseudoephedrine or Ephedrine could be extracted from various drug products containing Pseudoephedrine hydrochloride and can be converted into amphetamines. Amphetamines have potentially lethal stimulant effects on the central nervous system and heart and it is thereof important if such abuse potential could be minimized.

Pseudoephedrine HCl is a vasoconstrictor, which produces vasoconstriction by stimulating (alpha)-receptors within the mucous of the respiratory tract. Clinically Pseudoephedrine shrinks the swollen mucous membranes, reduces tissue hyperemia, edema and nasal congestion, and increases nasal airway patency. Its use is therefore significant in the relief from nasal congestion.

Pseudoephedrine HCl tablets used for the temporary relief of nasal congestion caused by common cold are commercially available in various strengths. However, soft gelatin formulations containing only Pseudoephedrine HCl and Guaifenesin as actives are not commercially available. The following table contains details of commercially available soft gelatin formulations comprising Pseudoephedrine HCl and Guaifenesin or Pseudoephedrine in combination of antihistamines or analgesics.

| Active Ingredient/s | |
|---|---|
| (Each Capsule contains) | Brand Name/Manufacturer |
| Guaifenesin 200 mg | Robitussin Cold & Cough/ |
| Pseudoephedrine HCl 30 mg | A. H. Robins |
| Dextromethorphan HBr 10 mg | |
| Pseudoephedrine HCl 30 mg | Nyquil/ |
| Doxylamine succinate 6.25 mg | Proctor & Gamble |
| Dextromethorphan HBr 10 mg | |
| Acetaminophen 200 mg | |
| Pseudoephedrine HCl 30 mg | Dayquil/ |
| Dextromethorphan HBr 10 mg | Proctor & Gamble |
| Acetaminophen 200 mg | |
| Pseudoephedrine HCl 30 mg | Alka-Seltzer Plus |
| Doxylamine succinate 6.25 mg | Night-Time Cold Medicine |
| Dextromethorphan HBr 10 mg | Bayer |
| Acetaminophen 325 mg | |
| Pseudoephedrine HCl 30 mg | Alka-Seltzer Plus |
| Chlorpheniramine Maleate 2 mg | Cold & Cough Medicine |
| Dextromethorphan HBr 10 mg | Bayer |
| Acetaminophen 325 mg | |
| Pseudoephedrine HCl 30 mg | Alka-Seltzer Plus |
| Chorpheniramine Maleate 2 mg | Cold & Cough Medicine |
| Acetaminophen 325 mg | Bayer |
| Pseudoephedrine HCl 30 mg | Alka-Seltzer Plus |
| Acetaminophen 325 mg | Cold & Sinus Medicine |
| | Bayer |
| Pseudoephedrine HCl 30 mg | Alka-Seltzer Plus |
| Dextromethorphan HBr 10 mg | Cold & Cough Medicine |
| Acetaminophen 325 mg | Bayer |

Pharmaceutical formulations comprising Pseudoephedrine HCl and Guaifenesin as principal ingredients are known. U.S. Pat. No. 5,141,961 to Coapman et al. describes a soft gelatin capsule comprising as a second pharmaceutical active, Pseudoephedrine HCl and Guaifenesin. This disclosure is directed to a highly concentrated liquid pharmaceutical composition solubilized using polyethylene glycol. The process therein described discloses the use of a solubilizing agents like polyvinylpyrrolidone or glycol for solubilizing the active ingredients.

U.S. Pat. No. 5,409,907 to Blase et. al describes a pharmaceutical suspension comprising a therapeutic amount of pharmaceutical active selected from the group consisting of acetaminophen, famotidine, pseudoephedrine hydrochloride, chlorpheniramine maleate, astemizole, dextromethorphan hydrobromide, guaifenesin, diphenhydramine hydrochloride, loperamide hydrochloride, simethicone, antacids, and combinations thereof. However, the suspending system described therein comprises an effective amount of xanthan gum and microcrystalline cellulose.

A composition including soybean oil, yellow beeswax and lecithin has been disclosed in the U.S. Pat. No. 6,309,667 to Horvath et. al. The patent does not disclose Pseudoephedrine HCl as an ingredient in combination with the other excipients.

U.S. Pat. No. 5,175,002 is directed to a suspension formulation comprising soybean oil, lecithin and wax. However the active in this formulation is Amantidine hydrochloride.

### SUMMARY OF THE INVENTION

It has been found that patient compliance is improved if a soft gelatin capsule is used for drug administration, because of its soft, elastic character which makes it easier to swallow when compared to conventional tablets or hard gelatin capsules. Furthermore, since the dosage form is generally swallowed without chewing, it is unnecessary to flavor or otherwise mask any unpleasant taste of the active pharmaceutical ingredients. Finally, unlike tablets, soft gelatin capsules do not chip or powder. Accordingly, we sought to devise a soft gelatin capsule formulation of Pseudoephedrine HCl because of these and other reasons.

In accordance with one preferred embodiment there is provided an orally administrable pharmaceutical formulation consisting essentially of an active pharmaceutical ingredient embedded into an oily matrix; an expectorant; a surfactant; a suspending agent; and a suspension medium.

In accordance with one preferred embodiment there are provided soft gelatin capsules of a pharmaceutical formulations consisting essentially of about 30.5 mg by weight of Pseudoephedrine HCl, about 200 mg by weight of guaifenesin, about 0.1-0.5 mg by weight of yellow beeswax, about 10-15 mg by weight of lecithin and about 200-300 mg by weight of soybean oil.

In accordance with another preferred embodiment there are provided methods of making a pharmaceutical formulation comprising the steps of preparing an oily matrix consisting of soybean oil and beeswax, blending lecithin to said oily matrix, adding guaifenesin to said matrix, mixing an active pharmaceutical ingredient into the said matrix and enclosing the oily matrix embedded pharmaceutical complex into a capsule, wherein Pseudoephedrine HCl is the active pharmaceutical ingredient. Preferably the amounts of each ingredient are as follows: about 30.5 mg by weight of Pseudoephedrine HCl, about 200 mg by weight guaifenesin, about 0.1-0.5 mg by weight of yellow beeswax, about 10-15 mg by weight of lecithin and about 200-300 mg by weight of soybean oil. In a preferred embodiment, the capsule is a soft gelatin capsule drug delivery device.

One possible advantage of preferred embodiments that the pseudoephedrine (either alone or along with one or more excipients) is coated with wax, making the possible extraction of Pseudoephedrine and its derivatives further difficult. Yet another advantage of preferred embodiments is that the drug delivery of the pharmaceutical formulation is achieved by a soft gelatin capsule and this makes it relatively difficult for someone to extract the active, unlike the case of a tablet as an OTC drug product. Hence the possibility of abuse of the drug is minimized.

In another possible advantage, preferred formulations have guaifenesin in combination with Pseudoephedrine HCl. Guaifenesin promotes lower respiratory tract drainage by thinning bronchial secretions, lubricates irritated respiratory tract membranes through increased mucous flow, and facilitates removal of viscous, inspissated mucus. As a result of pseudoephedrine and guaifenesin combination, sinus and bronchial drainage is improved, and dry, nonproductive coughs become more productive and less frequent.

Another possible advantage of preferred embodiments that preferred formulations include excipients like yellow beeswax and soybean oil, which are natural substances that make the extraction of Psuedoephedrine more difficult. This, in conjunction with the soft gelatin encapsulation, makes it a relatively complex multi-step process to extract amphetamines from the oily matrix. Thus preferred embodiments considerably minimize the potential to abuse the drug product.

### DETAILED DESCRIPTION OF THE PREFERRED EMBODIMENT

The present invention relates to pharmaceutical formulations having Pseudoephedrine, preferably Pseudoephedrine HCl, as an active ingredient for oral administration in the form of soft gelatin capsules. Preferred formulations also comprise guaifenesin, yellow beeswax, soybean oil and lecithin. In a preferred embodiment, the formulation consists essentially of the foregoing materials. We have used soybean oil in the preferred embodiment as a suspension medium and yellow beeswax as a suspending agent.

Preferred formulation includes guaifenesin that promotes lower respiratory tract drainage by thinning bronchial secretions, lubricates irritated respiratory track membranes through increased mucous flow and facilitates removal of viscous, inspissated mucus. As a result the sinus and bronchial drainage is improved and dry non-productive coughs become more productive and less frequent.

According to preferred embodiments, wax forms part of the fill composition that is inside the gelatin shell. The wax and oil mixture makes it difficult to isolate the active from the formulation.

The following examples illustrate preferred embodiments of pharmaceutical compositions comprising Pseudoephedrine HCl as principal ingredient.

### EXAMPLES

### Example 1

| | |
|---|---|
| Ingredients | Composition by weight |

| | | |
|---|---|---|
| Pseudoephedrine HCl, USP | 30.5 | mg |
| Guaifenesin, USP | 200 | mg |
| Yellow Beeswax | 0.1-5.0 | mg |
| Lecithin, NF | 10-15 | mg |
| Soybean Oil, USP | 200-300 | mg |

Although pseudoephedrine HCl is a preferred form of the pseudoephedrine, use of the free base or other salts of pseudoephedrine is also contemplated.

In general, gelatin capsule formulations for soft gelatin capsule comprise raw gelatin, plasticizer, solvent and optional ingredients such as flavors and colorants. Typically the plasticizer includes glycerin or sorbitol. A preferred plasticizer in this case is glycerin. One preferred gelatin formulation for the soft gelatin capsule used in accordance with preferred embodiment includes gelatin in the range of about 40-45% and a plasticizer in the range of about 18-25%. Capsule formulation can also include other suitable additives, which impart specific characteristics such as the look and feel of the capsule.

The following examples illustrate preferred embodiments of several soft-gelatin-shell Pseudoephedrine HCl/Guaifenesin formulations. These examples illustrate particular embodiments of the invention and are not intended to limit the scope of the invention in any way.

### Example 2

| Ingredient | Percentage by weight |
|---|---|
| Gelatin | 43.4% |
| Glycerin | 20.0% |
| Water | 36.6% |

### Example 3

| Ingredient | Percentage by weight |
|---|---|
| Gelatin | 58.5% |
| Glycerin | 31.5% |
| Water | 10.0% |

The various methods and techniques described above provide a number of ways to carry out the invention. Of course, it is to be understood that not necessarily all objectives or advantages described may be achieved in accordance with any particular embodiment described herein. Thus, for example, those skilled in the art will recognize that the formulations and methods may be formulated or performed in a manner that achieves or optimizes one advantage or group of advantages as taught herein without necessarily achieving other objectives or advantages as may be taught or suggested herein.

Furthermore, the skilled artisan will recognize the interchangeability of various features from different embodiments. Similarly, the various features and steps discussed above, as well as other known equivalents for each such feature or step, can be mixed and matched by one of ordinary skill in this art to perform methods in accordance with principles described herein.

Although the invention has been disclosed in the context of certain embodiments and examples, it will be understood by those skilled in the art that the invention extends beyond the specifically disclosed embodiments to other alternative embodiments and/or uses and obvious modifications and equivalents thereof. Accordingly, the invention is not intended to be limited by the specific disclosures of preferred embodiments herein, but instead by reference to claims attached hereto.

What is claimed is:

1. An orally administrable pharmaceutical formulation consisting essentially of an active pharmaceutical ingredient embedded into an oily matrix; an expectorant; a surfactant; a suspending agent; and a suspension medium.

2. The orally administrable pharmaceutical formulation according to claim 1, wherein the active pharmaceutical ingredient is Pseudoephedrine Hydrochloride.

3. The orally administrable pharmaceutical formulation according to claim 1, wherein the expectorant is guaifenesin.

4. The orally administrable pharmaceutical formulation according to claim 1, wherein the surfactant is lecithin.

5. The orally administrable pharmaceutical formulation according to claim 1, wherein the suspending agent is yellow beeswax.

6. The orally administrable pharmaceutical formulation according to claim 1, wherein the suspension medium is soybean oil.

7. An orally administrable pharmaceutical formulation consisting essentially of:
about 30.5 mg of Pseudoephedrine HCl,
about 200 mg of guaifenesin,
about 0.1-5.0 mg of yellow beeswax,
about 10-15 mg of lecithin; and
about 200-300 mg of soybean oil.

8. The orally administrable pharmaceutical formulation according to claim 7, wherein the formulation is disposed into a capsule.

9. The orally administrable pharmaceutical formulation according to claim 8, wherein the capsule is a soft gelatin capsule.

10. The orally administrable pharmaceutical formulation according to claim 7, wherein the surfactant is employed to provide lubricity to the matrix.

11. The orally administrable pharmaceutical formulation according to claim 10, wherein the formulation is disposed into a capsule.

12. The orally administrable pharmaceutical formulation according to claim 11, wherein the capsule is a soft gelatin capsule.

13. A process for preparing of an orally administrable pharmaceutical formulation comprising:
preparing an oily matrix comprising soybean oil and beeswax;
blending lecithin into said oily matrix;
adding guaifenesin to said matrix;
mixing an active pharmaceutical ingredient into said matrix; and
encapsulating the oily matrix-embedded pharmaceutical complex into a capsule.

14. The process for preparing of an orally administrable pharmaceutical formulation according to claim 13, wherein the active pharmaceutical ingredient is Pseudoephedrine hydrochloride.

15. The process for preparing of an orally administrable pharmaceutical formulation according to claim 13, wherein the capsule is a soft gelatin capsule.
